(19) United States
(12) Patent Application Publication
(10) Pub. No.: US 2016/0192897 A1
(43) Pub. Date: Jul. 7, 2016

(54) Stethoscope Chest Piece Finger Guard

(71) Applicant: Nicky Gazy, Scaggsville, MD (US)

(72) Inventor: Nicky Gazy, Scaggsville, MD (US)

(21) Appl. No.: 14/588,977

(22) Filed: Jan. 4, 2015

(51) Int. Cl. A61B 7/02

(52) U.S. Cl. CPC A61B 7/02

(57) ABSTRACT

In one example, we describe a method and system which consist of a small rubber attachment which can be slipped onto the chest piece of any stethoscope. We show designs, configurations, and descriptions of different finger guard designs. This reduces drastically or eliminates completely the background sound picked up from the fingers of the user/physician, which may interfere with diagnosing the physiologic sound picked up from the patient. In one example, we describe a method and system for Stethoscope Attachment, Auscultation Device. The device is a small attachment which can fit to any stethoscope. It connects between the removable chest-piece and tubing. The device houses a computer and sensor (e.g., membrane) which can receive incoming sound waves and analyze, or compare them to stored waveforms to categorize and analyze the incoming sound, to diagnose or recognize or classify the auscultated sound. Other variations are also shown.

### BACKGROUND OF THE INVENTION

The stethoscope is a useful auscultation device used often by physicians. However, it has some limitations regarding the clarity of the auscultated sound, which can result in an incorrect or limited diagnosis for the patient. When physicians use a stethoscope, the chest piece is held and pressed against the chest or any other area of interest. Unfortunately, the back of the chest piece (see image 1 in Appendix 4) also picks up sound from contact with the user's fingers. This picked up noise from the user's fingers is transmitted along with the physiologic sound from the patient making it sometimes difficult to distinguish the sound of interest. Without the background sound picked up from contact with the fingers, the sound being auscultated is much clearer. For this embodiment, we will show a solution that eliminates or reduces the background noise from contact with the stethescope chest piece drastically.

Another embodiment of this invention relates to a Stethoscope Attachment, Auscultation Device. The problem lies in that some physicians cannot always identify the exact sound they are hearing when auscultating the heart. Many physicians miss abnormal sounds on physical examination, and of those who are able to note abnormality, not all can accurately describe the sound. This is an issue as these sounds may be diagnostic of an underlying condition. Therefore, there is a need and market for this device, to be able to capture and correctly classify the sound wave form from the heart, for better diagnosis, with less error.

The invention and embodiments described here, below, have not been addressed or presented in any prior art.

### SUMMARY OF THE INVENTION

In one embodiment, we describe a method and system which consist of a small rubber attachment which can be slipped onto the chest piece of any stethoscope. Appendix 4 shows pictures and descriptions of the different finger guard designs. This reduces drastically or eliminates completely the background sound picked up from the fingers of the user. Thus, it is greatly improves the clarity of sound heard with respect to a very frequently-used device in the field.

In one embodiment, we describe a method and system for Stethoscope Attachment, Auscultation Device. The device is a small attachment which can fit to any stethoscope. It connects between the removable chest-piece and tubing. The device houses a computer and sensor, which can receive incoming sound waves and analyze, or compare them to stored waveforms to categorize and analyze the incoming sound. On the device, there is a digital panel which displays the analysis of the sound. The device gives users the option to switch between “Off” and “Auscultate”.

When the user switches the switch to “Auscultate”, the device will function as a normal stethoscope relaying sound heard at the chest-piece through the device into the stethoscope tube. When the user wants to analyze the heart sound, they first place the chest-piece on a new auscultation point and then press the reset/record button. The device will then pick up the incoming sound and will display the diagnosis. The device is able to match the sound within 1-4 waveforms. Of course, the more number of waveforms, the higher the accuracy of the determination and classification. When moving to the next point, the user will then first hit the reset/record, to erase the previous display, and then press the reset/record button once more, to record the sound from the new auscultation point.

### BRIEF DESCRIPTION OF THE DRAWINGS

FIG. 1 is one embodiment, as an example, for method of medical diagnosis.

FIG. 2 is one embodiment, as an example, for method of medical diagnosis.

FIG. 3 is one embodiment, as an example, for method of medical diagnosis.

FIG. 4 is one embodiment, as an example, for system of medical diagnosis.

FIG. 5 is one embodiment, as an example, for system of medical diagnosis.

FIG. 6 is one embodiment, as an example, for system of medical diagnosis.

FIG. 7 is one embodiment, as an example, for Stethoscope Chest Piece.

FIG. 8 is one embodiment, as an example, for Stethoscope Chest Piece Finger Guard.

FIG. 9 is one embodiment, as an example, for Stethoscope Chest Piece Finger Guard.

FIG. 10 is one embodiment, as an example, for Stethoscope Chest Piece Finger Guard.

FIG. 11 is one embodiment, as an example, for Stethoscope Chest Piece Finger Guard.

FIG. 12 is one embodiment, as an example, for Stethoscope Chest Piece Finger Guard.

FIG. 13 is one embodiment, as an example, for Stethoscope Chest Piece Finger Guard.

FIG. 14 is one embodiment, as an example, for Stethoscope Chest Piece Finger Guard.

FIG. 15 is one embodiment, as an example, for Stethoscope Chest Piece Finger Guard.

FIG. 16 is one embodiment, as an example, for Stethoscope Chest Piece Finger Guard.

FIG. 17 is one embodiment, as an example, for Stethoscope Chest Piece Finger Guard.

FIG. 18 is one embodiment, as an example, for Stethoscope Chest Piece Finger Guard.

FIG. 19 is one embodiment, as an example, for Stethoscope Chest Piece Finger Guard.

### DETAILED DESCRIPTION OF THE PREFERRED EMBODIMENTS

### Stethoscope Chest Piece Finger Guard:

We have multiple embodiments in this disclosure, related to Stethoscope for medical doctors. One embodiment is Stethoscope Chest Piece Finger Guard. Appendix 4 shows multiple designs and configurations/parameters for that embodiment.

Design 1 (configuration) Appendix 4 consists of a solid C shaped rubber piece which can be slipped onto the chest piece. The rubber piece fits the contours of the chest piece. To describe the rubber material, it would maintain its shape, yet it would be flexible enough to allow opening of the ends of the C to be slipped onto the stethoscope. The ends of the C could fit the contour of the nozzle for secure attachment, or leave an opening for the nozzle to provide the flexibility to be used on different stethoscope models. The rubber covering could extend to cover one half of the chest piece (extending to one auscultation end), or extend to both halves (providing noise reduction at both auscultation ends). Design 2 Appendix 4, as another example, is a solid O ring. This design would be ideal for the stethoscope chest piece with the arrow pointing to it. This would allow the user to remove the chest piece from the tubing of the stethoscope and slip on the O ring. The ring would be secured by the pressure between the small space between the nozzle and back of the chest piece.

Design 3 Appendix 4, as another example, is a large solid C shaped rubber piece. The rubber maintains its shape, yet is flexible to allow placement onto the chest piece. The difference between this design and Design 1 is the amount of space left between the two sides of the chest piece. Again, the ends of the C would fit the contour of the nozzle, or provide an opening as in Design 1.

Image B Appendix 5, as another example, is to create a stethoscope chest piece, but rather than the back consist only of metal, it would have a thin layer of built-in rubber to cover the metal and provide protection against noise. This rubber cover is not removable, as initially manufactured. In this way, the fingers are contacting rubber, not metal.

For Design 4 for the Stethoscope Chest piece Finger Guard, as shown in image A Appendix 5, as an example, the chest piece is made of the metal with two auscultation ends. The ends have a plastic cover over them (the cover with “L”), and these covers are held in place by a black rubber ring. Design 4 consists of a chest piece where the rubber noise guard is built onto the chest piece itself, making contact only with the exposed metal area in the image. This rubber guard would allow space for the rubber rings at either end to be removed. For example, the chest piece would look like image B of Appendix 5, with rubber coating rather than black metal finish, as shown in the image.

Now, take, for example, Design 4 Appendix 4, shown above. The rubber could have two parts: a hard yet flexible outer portion, and a malleable or soft inner portion, to get the shape of any contour. This would allow the outer portion of the finger guard to maintain its shape when fastened to a chest piece, and the inner portion to be soft enough to fit the contour of many stethoscope chest piece designs. The soft portion also reduces finger or background noise transmission to the listening device. The material described here can be applied to any shape taught in this disclosure.

In one embodiment, we use soft plastic, hard plastic, bendable metal sheet or band, such as titanium or copper or tin or steel, elastic band, rubber, playing clay, play dough, cotton, wool, synthetic materials, silicone, fiber, clothing, carbon fiber, elastic material, soft wood, or wood products. In one embodiment, we use multiple bands, in parallel or in series, or on top of each other. In one embodiment, we use C shape, or O shape, or ( ) shape, or ( ) shape (i.e., with a larger gap between 2 pieces). In one embodiment, we use glue to attach it to the metal or device below. In one embodiment, we use band or buckle or Velcro or shoe lace or string or chain or cable or twisted wire or wire. In one embodiment, we use synthetic material, cotton, metal, alloy, wool, fabric, leather, or the like, for one or more layers, bands, or rings, or combination of them. In one embodiment, it fits different shapes and sizes of the device, within a reasonable range, with its elasticity or rubber quality.

In all of these, the background noise or sound from finger or otherwise is reduced or eliminated, for much better sound detection, with better diagnosis, for better treatment of the patient by doctors, which is a major improvement for our device here, in medical field or other fields, e.g. for heart or other parts of the body, for humans and animals, or for equipment or the like.

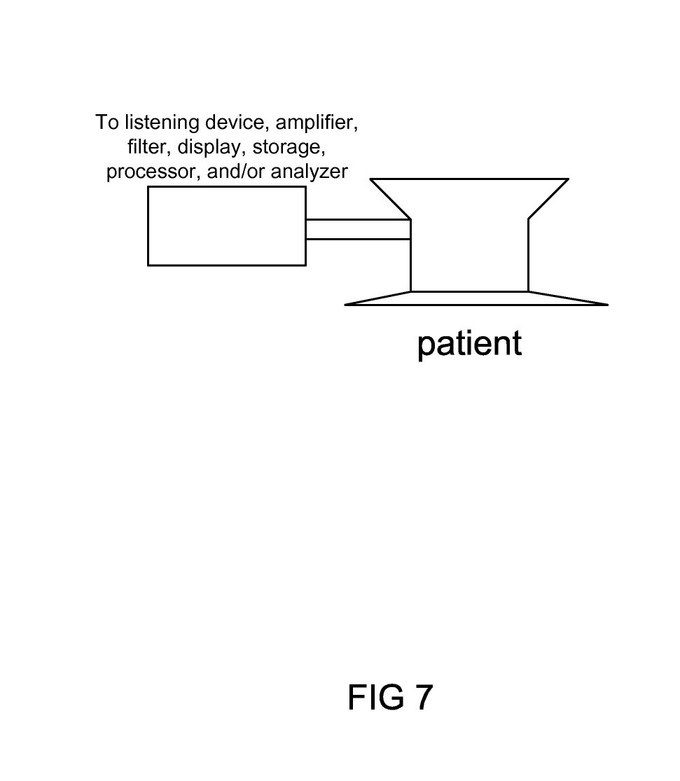

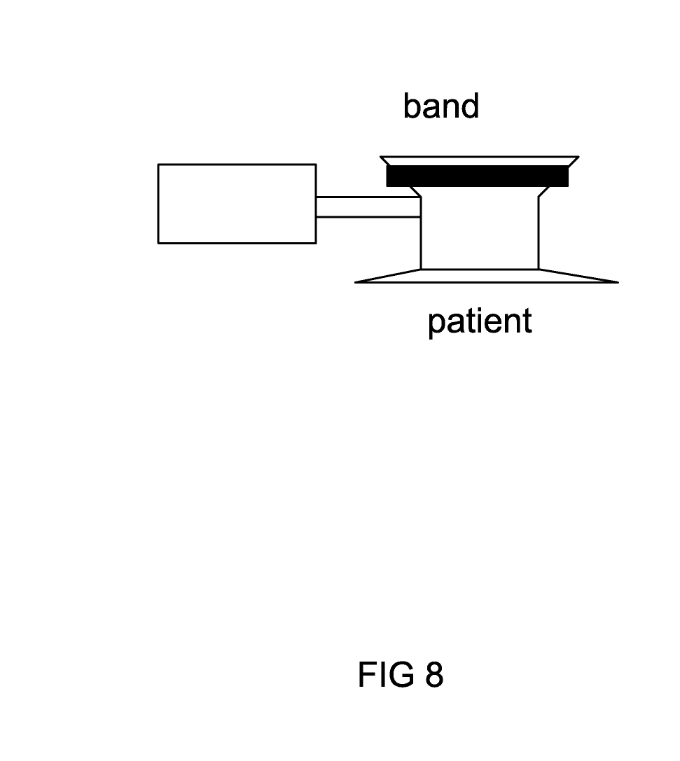

FIG. 7 is one embodiment, as an example, for Stethoscope Chest Piece, which is connected to one or more of the following: listening device, speaker, amplifier, recorder, storage, memory, filter, display, processor, or analyzer, or a combination of the above. FIG. 8 is one embodiment, as an example, for Stethoscope Chest Piece Finger Guard, with a band on top only.

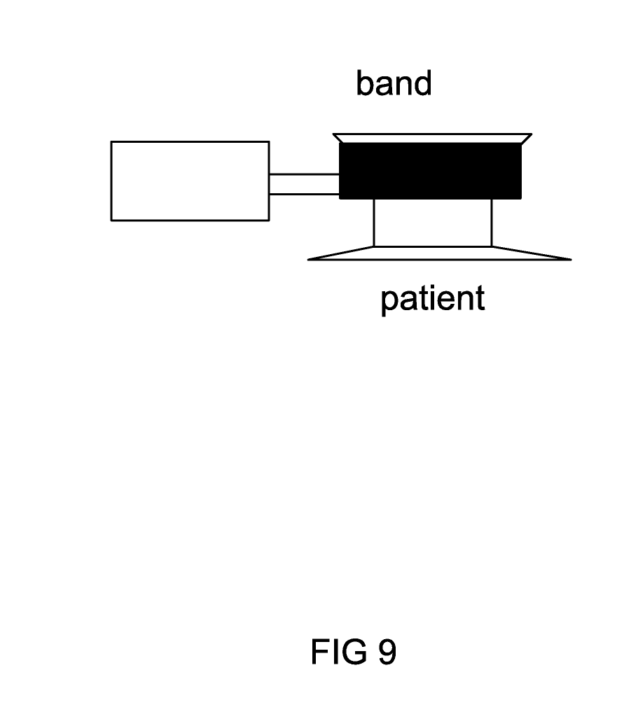

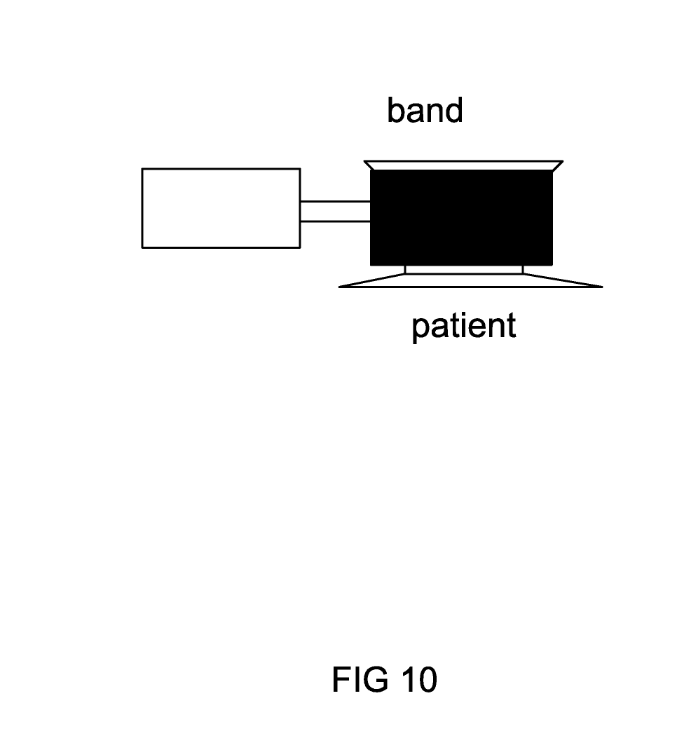

FIG. 9 is one embodiment, as an example, for Stethoscope Chest Piece Finger Guard, with a band on top and mid-section, only. FIG. 10 is one embodiment, as an example, for Stethoscope Chest Piece Finger Guard, with a band on top and mid-section, as well as lower section.

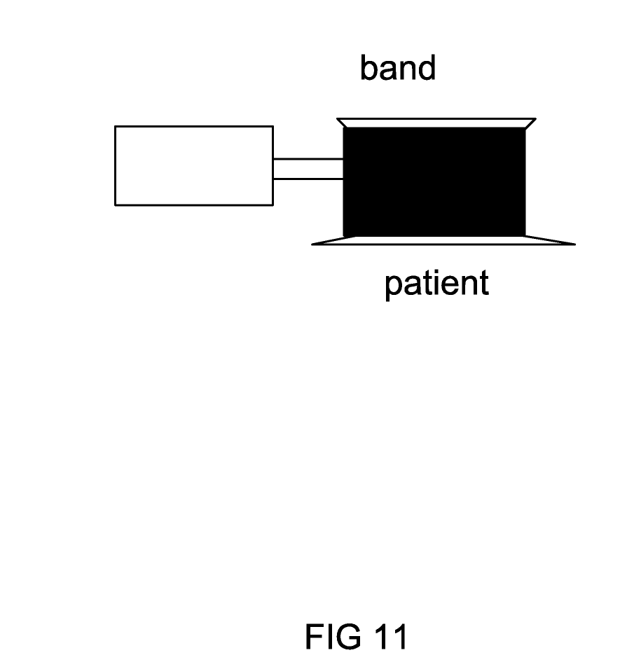

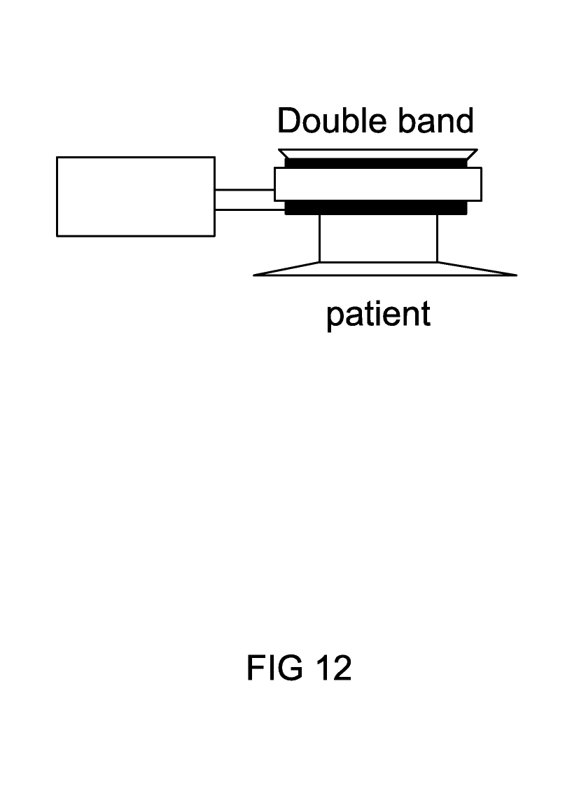

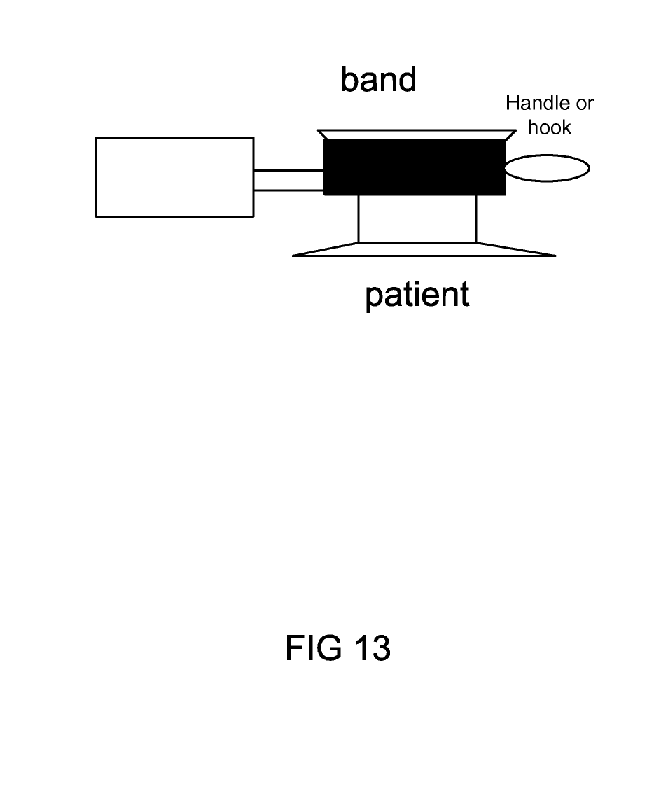

FIG. 11 is one embodiment, as an example, for Stethoscope Chest Piece Finger Guard, with a band on top and mid-section, as well as lower section and bottom section. FIG. 12 is one embodiment, as an example, for Stethoscope Chest Piece Finger Guard, with a double band, on top of the first band. FIG. 13 is one embodiment, as an example, for Stethoscope Chest Piece Finger Guard, with a band with a handle or hook, for the user, to remove that or attach it to a hook.

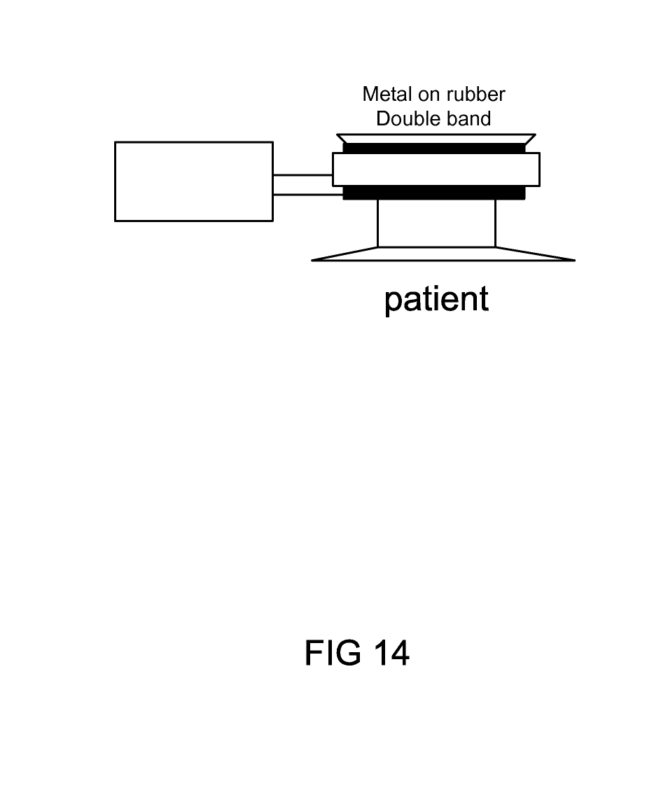

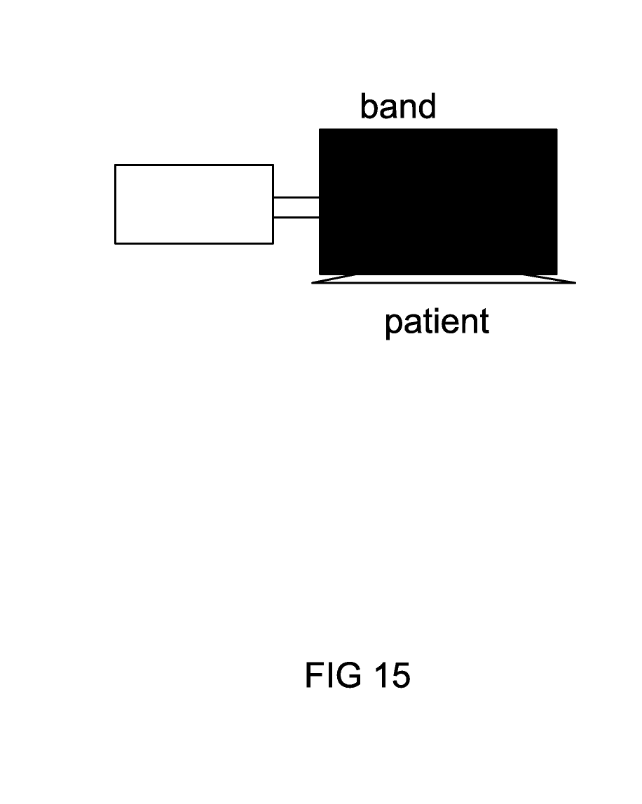

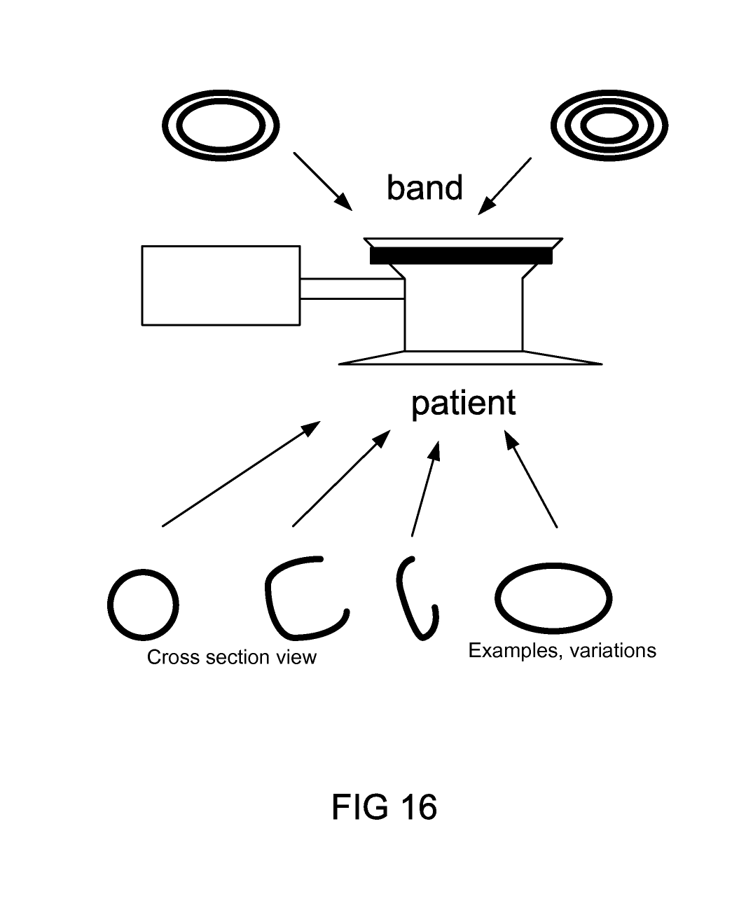

FIG. 14 is one embodiment, as an example, for Stethoscope Chest Piece Finger Guard, with double-band, with metal on rubber, or any other combination of materials as mentioned in this disclosure. FIG. 15 is one embodiment, as an example, for Stethoscope Chest Piece Finger Guard, with a thick band covering everything. FIG. 16 is one embodiment, as an example, for Stethoscope Chest Piece Finger Guard, with various variations and examples of bands or rings O-rings or washers or multi-bands or partial-curves or semi-circles.

In one example, the C-shaped plastic or metal band has a spring action, for opening and closing the opening on the C shape, for installing and removing the band. In one example, the soft material on the ring or band or multi-band, in contact with the chest piece, keeps the vibration or noise to the minimum, as it kills/damps all the vibration quickly/exponentially, e.g., using gelatin or play dough-type material, or multiple materials sandwiched together as multi-layered.

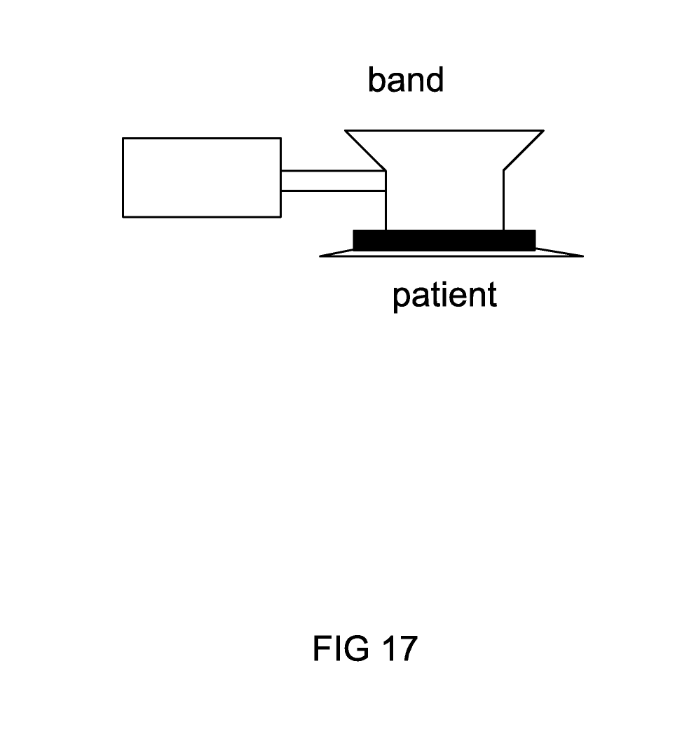

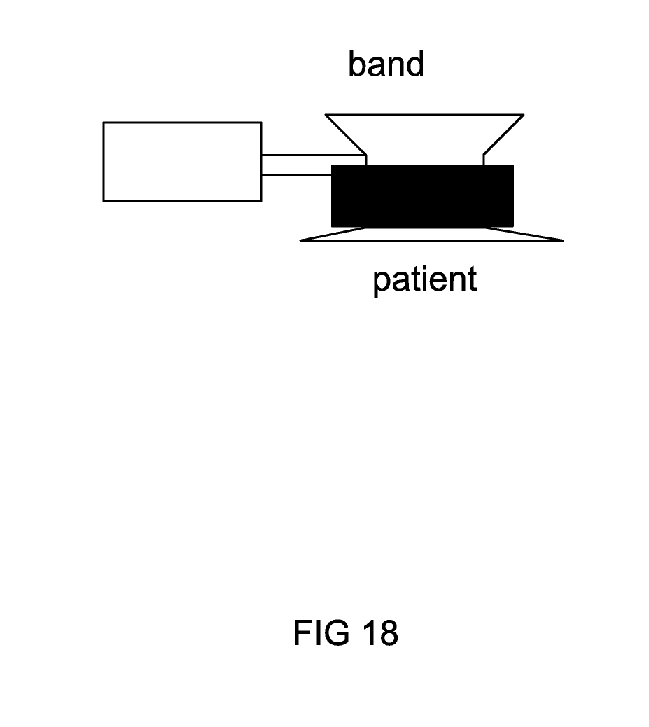

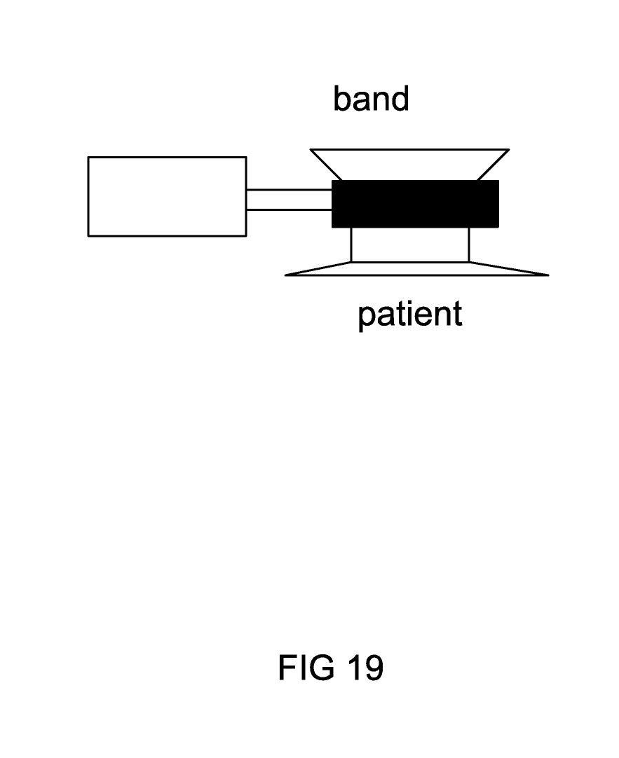

There are other variations of the setup for the invention, e.g.: FIG. 17 is one embodiment, as an example, for Stethoscope Chest Piece Finger Guard, similar to that of FIG. 8, but from opposite side. FIG. 18 is one embodiment, as an example, for Stethoscope Chest Piece Finger Guard, similar to that of FIG. 9, but from opposite side. FIG. 19 is one embodiment, as an example, for Stethoscope Chest Piece Finger Guard, in the middle, using a notch or gap to accommodate the extension element or tube, which is connected to the chest piece, located on the side of the extension element or tube.

### Stethoscope Attachment, Auscultation Device:

In one embodiment, we describe a method and system for Stethoscope Attachment, Auscultation Device. The device is a small attachment which can fit to any stethoscope. It connects between the removable chest-piece and tubing. The device houses a computer and sensor which can receive incoming sound waves and analyze, or compare them to stored waveforms to categorize and analyze the incoming sound. On the device, there is a digital panel which displays the analysis of the sound. The device gives users the option to switch between “Off” and “Auscultate”.

The device classifies based on template and waveforms, or matching to known patterns, for the waveforms, which then signifies the pattern of sound made by the heart. From the signatures and envelops, or peaks and valleys, or ratios of maxima and minima, or distances for peaks, based on point-by-point comparison, as an example, or general envelop coverage, e.g., as normalized, to match the shapes and peaks, to find the best match or closely-matched candidate, for presenting the result to the doctor, with corresponding correct heart sound or likely heart sound, for the patient.

The conventional stethoscope has a chest piece with diaphragm to pick up the sound from the chest, which sends the sound via a tube to the ear of the doctor, with no electronics in between. As shown in Appendix 2 page 1, we have options for ON (electronic components will function) and OFF (electronic components will not function, yet sound will still be transmitted through the device). The chest piece is connected to a tube or extension, via a nozzle, which is removable, which ends up with a listening device for 2 ears, or speaker, or amplifier. The “Stethoscope Attachment, Auscultation Device”, shown on top-middle section of the Appendix 2 page 1, sits between the extension/tube on the left side and chest piece on the right side. This can also display the waveform directly for the user.

The “Stethoscope Attachment, Auscultation Device” has a LED-display (or any other type of display), record/reset button, tube on right side, and the nozzle on left side. It analyzes the sound waves or raw data and displays the sound waves or raw data. There can potentially be a membrane sensor or any other type of sensor in the Stethoscope Attachment, Auscultation Device to pick up the sound waves. This sensor would be connected to a processor or computer. In one case, the chest piece and the Stethoscope Attachment, Auscultation Device are one unit, and in another case, they are separate and changeable. (See page 2 Appendix 2.)

Appendix 1 shows crescendo, decrescendo, crescendo-decrescendo, and uniform patterns, as well as different heart sounds, including PSM, ESM, HSM, MSM, LSM, EDM, MSC, Split S2, EDS, OS, S3, S4, Split S1, and ES. The factors for the results are: the timing of the heart sound and characteristic of the waveform.

The intensity for hearing the result is measured based on, e.g., scale of 1 through 6, e.g., where 1 means barely-heard, 5 means very loud, and 6 means that one can hear without the stethoscope. The scale is very subjective and not be able to calibrate at all with the conventional devices. However, with our normalization and peak intensity measurements, we can introduce the scale from 0 to 1, or 0 to 100, or percent, to meaningfully and mathematically quantify and measure the strength of the waveforms and peaks/intensities. So, that is a strong result for calibration, using our method.

The device is able to diagnose heart sounds. (Please note that the peaks 1 and 2 shown in figures signify S1 and S2, which are taken from a reference website, as noted.) It will also be able to diagnose extra sounds such as an S3 or S4 sound (S3, S4), ejection sound (ES), mid-systolic click (MSC), early diastolic sound (EDS), and an opening snap (OS). The device will also be able to classify the type of murmur heard. Murmurs will be classified as late systolic murmur (LSM), pre-systolic murmur (PSM), early systolic murmur (ESM), early diastolic murmur (EDM), mid-systolic murmur (MSM), mid-diastolic murmur (MDM), continuous murmur (CM), or holo-systolic murmur (HSM).

The device is able to distinguish all sounds, and combinations of sounds (such as LSM/S2). Should the device not be able to distinguish the sound, it will give an error interval (e.g., “error at late-systolic to mid-diastolic” interval).

Design 1 (Configuration):

From the tube to nozzle of the device, there will be one continuous path. The sound will pass from the chest-piece to the membrane (or sensor), through the membrane into the nozzle, and then into the tube of the stethoscope.

Design 2:

The sound will travel from the chest-piece to the tube of the device where it enters. The sound wave will then enter the device and hit the membrane where it is picked up and goes to a transducer. From there, the signal splits and is analyzed to be displayed on the digital screen, as well as being sent to a speaker which is housed in the end of the device. From there, the speaker will emit the sound and the sound will be sent to the tubing of the stethoscope towards the user's ears.

Design 3:

The chest piece and device are one piece. Within this design, Design 1 or 2 can be implemented.

Design 4:

The entire stethoscope including tubing, ear tube and tips, device, and chest piece are one piece. The tips can be removable for one example.

### Details of the Invention: Heart:

It is best to imagine the heart as a 2×2, 4 chambered pump. (The 2 chambers at the top usually pump essentially at the same time, and the 2 chambers at the bottom usually pump essentially at the same time.) There are one-way valves (in a normal heart) between the top and bottom chambers allowing flow to go from top to bottom. For our purposes, we will imagine the two sets of pumps (top/bottom on left, and top and bottom on right) to be separate. The bottom pumps each has another one-way valve attached to them allowing ejection of fluids.

Heart Auscultation:

Auscultation means listening to the body sounds using a stethoscope. When auscultating the heart, the user places the chest-piece over a point of interest. In the normal heart, usually, two sounds are heard, the S1 and S2. S1 is heard when the bottom chambers pump causing the valve between the top and bottom chambers to shut, creating the S1 noise. For our purposes, S2 is heard after the bottom pumps pump their fluids and the valve allowing ejection of fluids from the bottom pumps closes, creating the S2 noise. From S1 to S2 is the systolic phase, and from S2 to the next S1 is the diastolic phase.

In a pathologic heart, there can be issues with the valves, musculature, vasculature, or walls of the chamber. These defects can create additional sounds which are of interest to the device.

Appendix 1 shows a lot of examples for various shape forms and waveforms with various peaks and signatures, as discussed above, for various problems with the heart. It also references some articles which they came from, e.g., from Internet websites.

Appendix 3 shows 3 variations of the designs and configurations for the setup for the inventions, called Design 1 through Design 3 (configuration), where the placement of the components are changed, as shown in the figures explicitly, for Design 1, Design 2, and Design 3 (configuration).

Appendix 3 Design 1 shows a membrane in the tube, with complete tube, so regardless of ON/OFF, one can still hear transmitted sound from the chestpiece diaphragm. In Design 2, there is a split, with a speaker or amplifier after the split or membrane, with one going for tube to listening device, and one going to analyzing section for sound analysis by computer. Designs 1 and 2 can be removable. In Design 3, the device and chest piece are in one piece/integrated/built-in.

In other embodiments, one can have tube, membrane, speaker, listening device, amplifier, ear piece, chest piece, and the “Stethoscope Attachment, Auscultation Device”, shown in Appendix 2, as one piece, two pieces, or multiple-piece devices, with one or more components (mentioned in the list above) integrated with each other, as one piece or built-in.

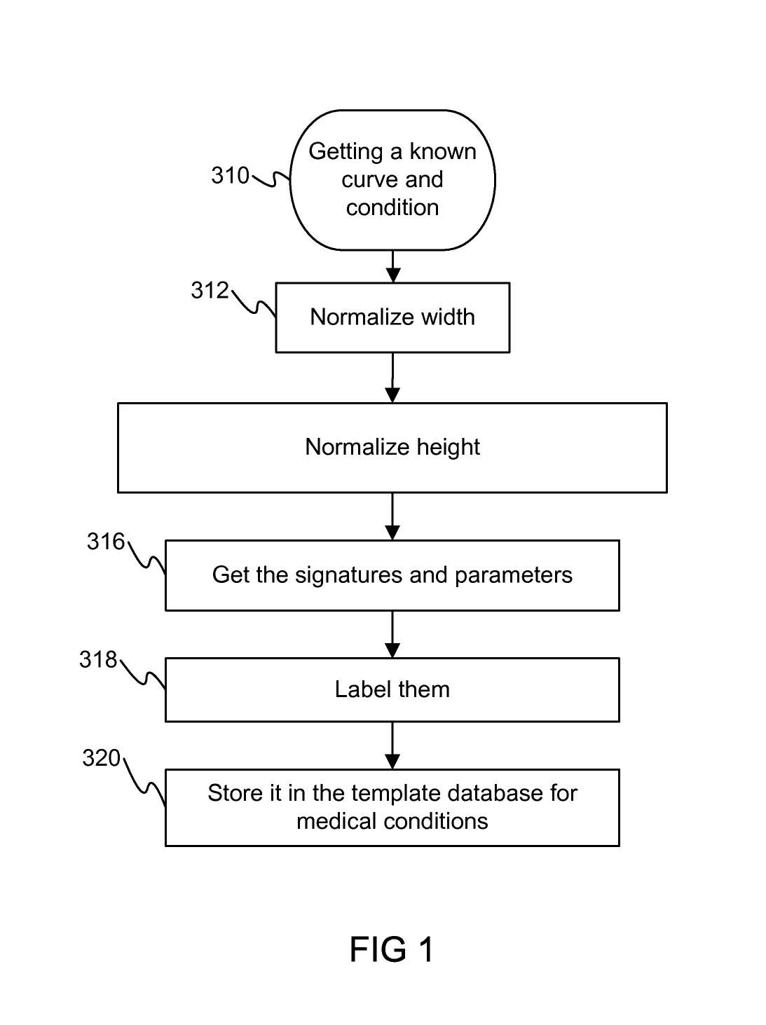

FIG. 1 is one embodiment, as an example, for method of medical heart sound diagnosis. It gets the curve for an unknown condition, normalizes for width and height, gets the signatures and parameters, labels them, and stores them in templates database for later comparison.

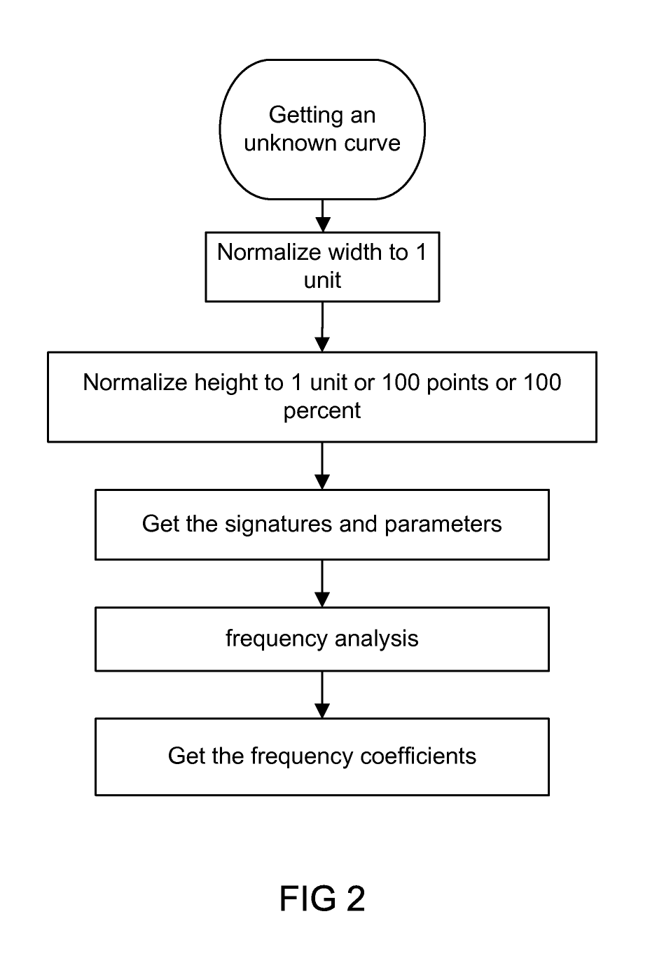

FIG. 2 is one embodiment, as an example, for method of medical heart sound diagnosis. It normalizes the curves in 2 directions, X-Y coordinates, based on the e.g., max-min distance for the curves, in that coordinate, normalized to value 1 or 100, or 100 percent, with that ratio, for the whole range, to scale the values, to be able to compare with template curves in database, which are already normalized. For example, if there are 250 mm between max-min distance for the curves, for peak-to-valley max distance, then we set that 250 mm=1 unit normalized. So, each 1 mm on that coordinate will be equal to (1/250) of a unit normalized. All other distances in that direction/coordinate will be scaled with that ratio. The same thing for the X-range on other direction or width can be done to get another scaling factor for the width or X-direction. The 2 scaling factors make it possible to compare curves point-by-point, with each other or with templates.

For a point within 5 percent (or a threshold percent) of another coordinate value, of another point in a template, we call it an overlap, or match, for a point. So, if we have enough matches for number of points between 2 curves, e.g., above 85 percent match on total number, then the 2 curves are matched. Then, the corresponding condition or signature for the curve, for the template matched, will be shown to the doctor or user, as the heart sound diagnosis for the patient.

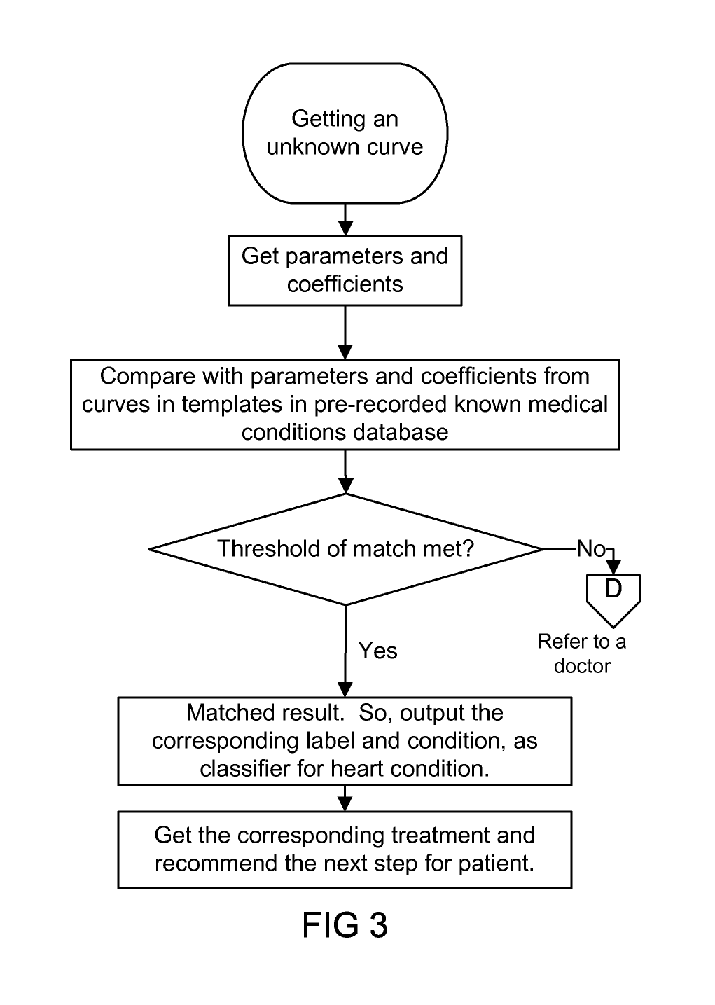

It can also do frequency analysis, e.g., Fourier analysis, as well as other signatures and features on the curves, or other parameters. FIG. 3 is one embodiment, as an example, for method of medical heart sound diagnosis, using the matched threshold, e.g., 85 percent of the points are matched, to have a matched curve. If there is not enough for a match, then it refers to a human or doctor, for manual evaluation of the patient.

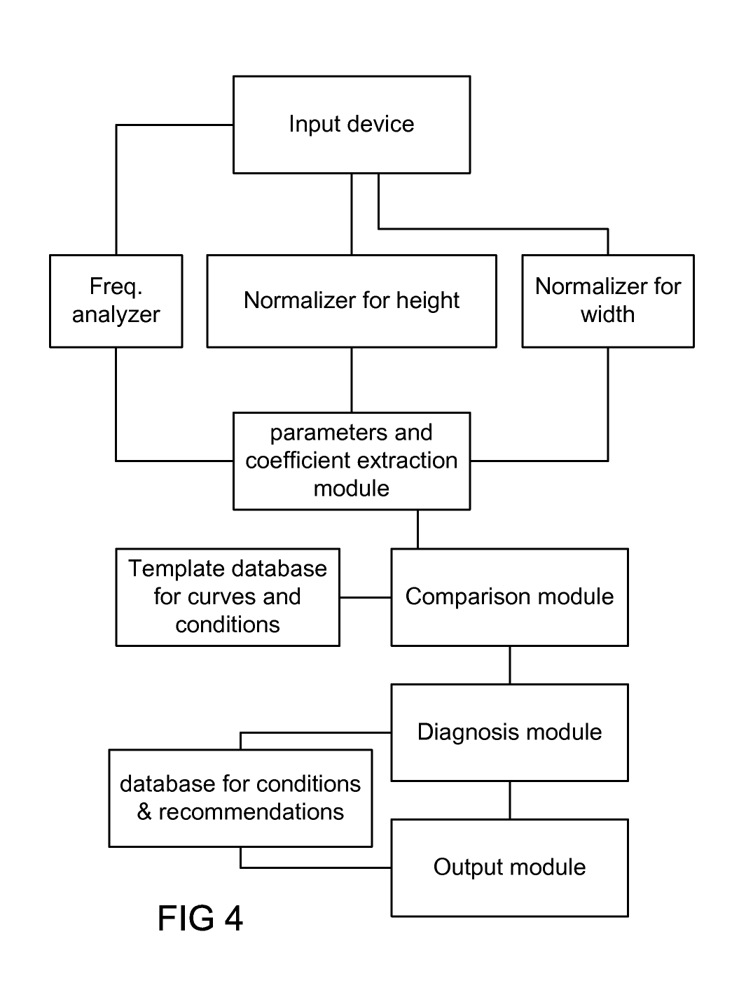

FIG. 4 is one embodiment, as an example, for system of medical heart sound diagnosis, with input device, for listening mode, normalizer modules, freq. analyzer, or oscilloscope, coefficient extraction module, comparison module, sound diagnosis module, and output module, e.g., display/monitor of the computer, along with supporting databases for curves and recommendations.

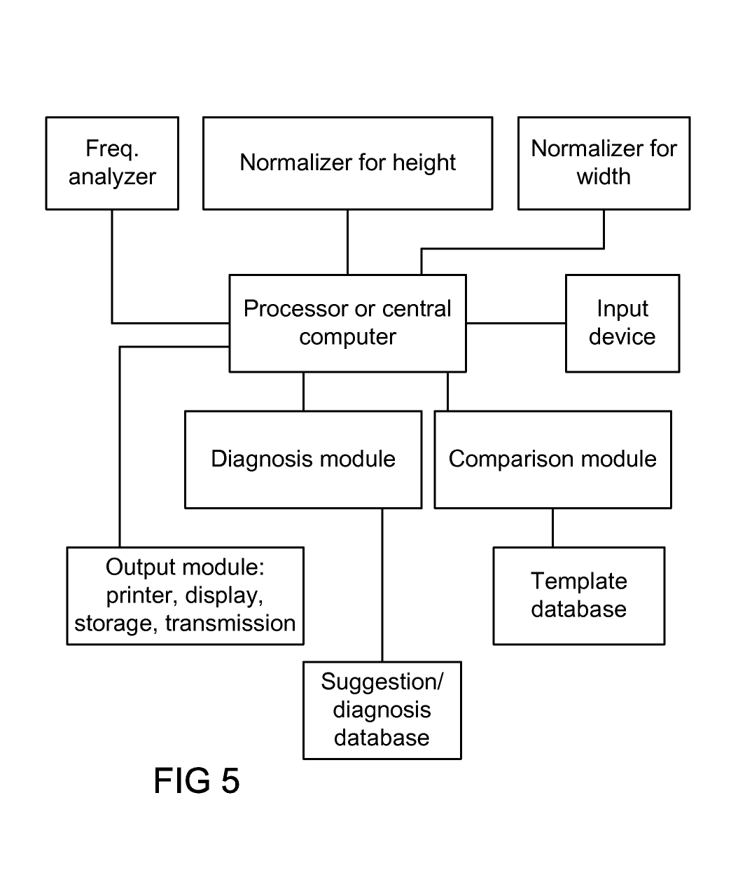

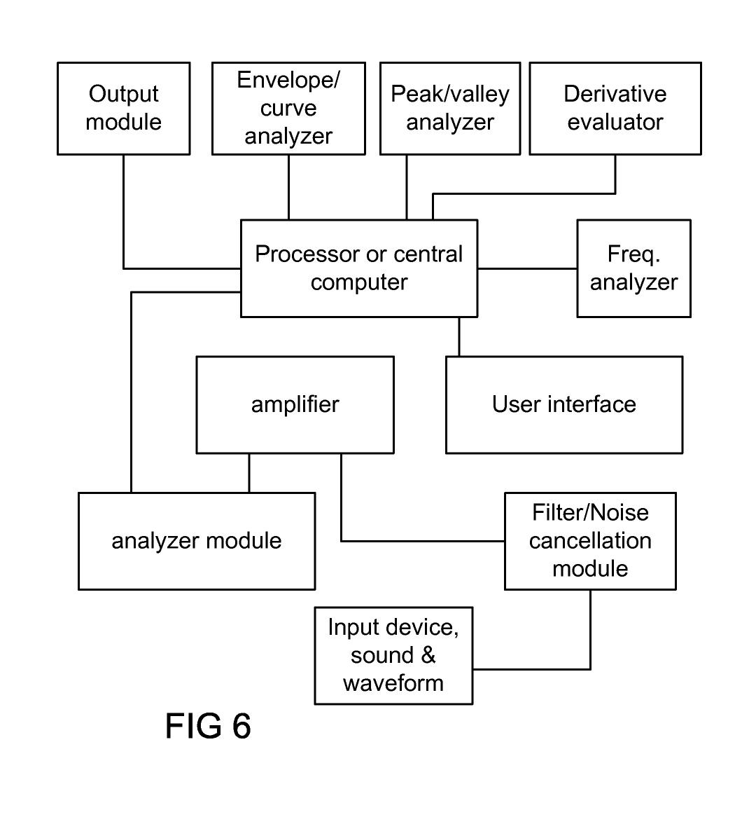

FIG. 5 is one embodiment, as an example, for system of medical heart sound diagnosis, with server or server farm or central computer or distributed processors. FIG. 6 is one embodiment, as an example, for system of medical heart sound diagnosis, with envelope analyzer, peak-valley analyzer, derivative-slope analyzer, as well as user interface for user input, plus filters and amplifiers for pre-processing for better input signal or data, in addition to digitizer or quantizer for conversion to digital data, which is well-known in the art, and thus, is not described here anymore.

The way we compare the curves, in one embodiment, we normalize the curves in height (of the biggest peak) and width of the waveform, to be able to match the normalized database library for those situations mentioned above, as shown for example in Appendix 1, which were already pre-recorded and tabulated in our database for comparison, to match with heart sound diagnosis.

To get the match, we say it is a match, if, for example, a percentage or ratio of the points on the curves match each other or overlap, e.g., if more than 80 or 75 percent matches, we say that the curves is matching the template, e.g., for PSM or ESM, as shown in Appendix 1, and output those (PSM or ESM) as the diagnosis of the heart sound, in the machine/device for the heart monitor.

Another way to match a curve with template is by envelop-matching of 2 curves or waveforms, with some threshold of matching the envelops, as described above, point-by-point, e.g., if over 80 percent of the points match each other, calling it as “matched”, with the template and corresponding heart sound, which will be recorded and outputted on the device or monitor or display or printout.

Another way to match/compare a curve with a template in database for a condition is by Fourier analysis of the frequency component and coefficients corresponding to the curves, so that if enough coefficients match in the frequency domain, e.g., more than 75 percent, then we call it a “match”. Then, we output the corresponding heart sound diagnosis.

Another way to match/compare a curve with a template is the signature of the highest peak, size, location, relative location for the peak, ascending curve, descending curve, slope of the curve, envelop for the curve, how fast the peaks oscillate, number of peaks, frequency of the peaks, peak to valley ratio, first derivative of the curve, 2^{nd} derivative of the curve, or other parameters obtained from the curve. If the majority of the parameters, obtained for comparison, e.g., above 80 percent, are the same, or about the same, within a margin of error, e.g., 5 percent different, or within some absolute number for the difference, then we call it a “match”. Then, we output the corresponding heart sound diagnosis.

If more or new heart sounds are classified or entered into the database for templates, at a later time, then the comparison and sound diagnosis will be more comprehensive and complete, with better and more detailed results and conditions.

In one embodiment, we display and output the actual waveform which the device picks up for the doctor to see. In one embodiment, we have the wave samples for “pericarditis” as one of the heart sounds which would be analyzed or detected. In one embodiment, we have the device pick up/record “beats per minute” (bpm), as well, as a parameter, for comparison for doctor for diagnosis. In one embodiment, we have the device fit into the size of about 7.5×2.5×1.5 cm, as an example.

In one embodiment, we have the device pick up lung sounds. The lung sounds are the sounds which are heard when auscultating (listening) to the chest during inspiration and expiration. In one embodiment, in order to incorporate this into the device, the “Cardio/Off Switch” should be changed to the “Cardio/Resp/Off Switch”. As an example for lung sound reference guide, please refer to the Internet guide: http://www.easyauscultation.com/lung-sounds-reference-guide

Thus, this device can be used on the lung and other parts of the body or physical objects for diagnosis of the sound wave. For the band, we can have any shape such as polygon, or any non-uniform or soft boundary shapes, e.g., to get or fit the contour of the fingers or being flexible to the touch, and using any materials or combination of the materials as mentioned above. In one example, rubber or elastic or soft material are used for low transmission of sound or fast dampening of the sound waves within short distances in the material. To that effect, having multiple boundaries or layers helps dampen the sound waves at the interfaces.

Any variations of the above teaching are also intended to be covered by this patent application.

1. A stethoscope medical listening device, said device comprises:
a chest piece;
a finger guard;
an extension;
wherein said extension is attached to said chest piece;
wherein said extension is connected to a listening device;
wherein said finger guard is attached to said chest piece;
wherein said finger guard is located around said chest piece;
wherein said finger guard is in shape of a complete full circle, with 360 degrees coverage on circumference of said circle, as one-piece, made of rubber or elastic material;
wherein said finger guard is located above or below of said extension, with respect to said chest piece;
wherein said finger guard is a removable piece, with respect to said chest piece.

2. The stethoscope medical listening device as recited in claim 1, wherein said finger guard is a double-band.

3. The stethoscope medical listening device as recited in claim 1, wherein said finger guard is a multiple-band.

4. The stethoscope medical listening device as recited in claim 1, wherein said extension is connected to an amplifier.

5. The stethoscope medical listening device as recited in claim 1, wherein said extension is connected to a filter.

6. The stethoscope medical listening device as recited in claim 1, wherein said extension is connected to a computer or processor.

7. The stethoscope medical listening device as recited in claim 1, wherein said extension is connected to a display or monitor.

8-20. (canceled)
